(19) United States
(12) Patent Application Publication
(10) Pub. No.: US 2026/0347764 A1
(43) Pub. Date: Nov. 5, 2026

(54) AN IN VITRO METHOD FOR PRODUCING A 3D-STRUCTURE OF CELLS

(71) Applicant: Universiteit Maastricht, Maastricht (NL)

(72) Inventors: Erik Jacob VRIJ, Maastricht (NL); Laury Amelia ROA FUENTES, Maastricht (NL); Stefan GISELBRECHT, Maastricht (NL); Clemens Antoni VAN BLITTERSWIJK, Maastricht (NL)

(21) Appl. No.: 19/121,231

(22) PCT Filed: Oct. 20, 2023
(86) PCT No.: PCT/NL2023/050556

§ 371 Date: Apr. 15, 2025

(30) Foreign Application Priority Data
Oct. 21, 2022 (EP) 22203142.9

(51) Int. Cl. C12N 5/00

(52) U.S. Cl. CPC C12N 5/0062; C12N 2500/90; C12N 2501/01; C12N 2501/119; C12N 2501/385; C12N 2501/727

(57) ABSTRACT

The invention concerns an in vitro method for producing a 3D structure of endoderm cells, mesoderm cells and ectoderm cells and capable of further organogenesis to form an organ or organ-like cell structure. The method comprises seeding one or more pluripotent stem cells in a culture chamber containing culture medium, followed by culturing the seeded cell or cells in culture medium comprising an amount of chemical inducers for forming a plurality of pluripotent stem cells and inducing part of the formed pluripotent stem cells to form extraembryonic stem cells to obtain a primitive endoderm/epiblast aggregate. The aggregate is cultured in fresh culture medium to obtain an extraembryonic endoderm/epiblast rosette structure. Culture medium is flushed from the extraembryonic endoderm/epiblast rosette structure and the extraembryonic endoderm/epiblast rosette structure is cultured in fresh serum free culture medium comprising a reducing agent to obtain the 3D structure of cells.

The invention relates to an in vitro method for producing a 3D structure of endoderm cells, mesoderm cells and ectoderm cells and capable of further organogenesis to form an organ or organ-like cell structure. The invention further relates to culture media that enable such in vitro method for producing a 3D structure of endoderm cells, mesoderm cells and ectoderm cells and capable of further organogenesis to form an organ or organ-like cell structure.

Three-dimensional structures of endoderm, mesoderm and ectoderm cells hold high potential value. For example, such a multicellular 3D structure may be derived from a mammalian embryo, e.g. by harvesting either stem cells, germ-line cells or larger multicellular parts from the embryo. The 3D structure of cells can be used for research purposes such as investigating mammalian embryonic development or drug discovery or drug testing, or be used for product development such as in regenerative medicine, for example growing implantable cell tissue or even whole organs from the 3D cell structure.

A major concern with harvesting the needed parts of the mammalian embryo to obtain the three-dimensional structures of interest is that the embryo usually is prevented from becoming a live animal and the pregnant mammal carrying the embryo often has to be sacrificed. As a consequence a lot of effort has been put in developing methods to produce the desired 3D structures without having to harvest embryos from pregnant animals.

The early mammalian conceptus consists of three cell lineages, the pluripotent epiblast (Epi), which forms the embryo proper, and two extra-embryonic lineages, the trophoblast and primitive endoderm (PrE), that contribute to the placenta and yolk sac, respectively. In order to form 3D structures of cells that mimic the early stages of mammalian embryos, one known way of producing such 3D structures of cells is thus to grow pluripotent stem cells, trophoblast stem cells (TSCs) and extraembryonic endoderm stem cells in combination. The stem cells can proliferate, differentiate and organize into most embryonic tissue, but the known method still requires the use of embryo derived trophoblast cells. This may raise ethical concerns amongst others, and moreover complicates the method in view of having to maintain different cell lines. It has also been shown that the formation of blastocyst-like structures is induced by combining trophoblast stem cells and embryonic stem cells (ESCs), which structures were termed blastoids. Blastoids generate PrE-like cells from the ESCs, thus providing the three cell lineages of the mammalian conceptus. However, also in this case the use of trophoblast cells is necessitated.

It is accordingly an aim to provide an in vitro method for producing a 3D structure of endoderm cells, mesoderm cells and ectoderm cells and capable of further organogenesis to form an organ or organ-like cell structure without having to use trophoblast cells.

In mice, the bifurcation between PrE and Epi cells is established between E3.25 and E4.5 (Schrode et al., 2014; Onishi and Zandstra, 2015; Chazaud et al., 2006; Bassalert et al., 2018; Plusa et al., 2008), and is marked by the timed expression of the transcription factors Oct4, Nanog, Klf4 and Sox2 in the Epi (Neagu et al., 2020), and Gata6, Pdgfrα, Gata4, Sox17 and Sox7 in the PrE (Lokken and Ralston, 2016; Artus et al., 2013; Lo Nigro et al., 2017). Experiments suggest that PrE specification is initiated by lineage priming (Ohnishi et al., 2014) that exploits polycomb (Illingworth et al., 2016), chromatin modifier (Goolam and Zernicka-Goetz, 2017) and small-RNA (Ngondo et al., 2018) activities, along with the progression of gene regulatory networks (Lokken and Ralston, 2016) and intercellular signaling circuitries [e.g. FGF/Mapk/Erk (Azami et al., 2017; Kang et al., 2017; Molotkov et al., 2017; Ohnishi et al., 2014; Krawchuk et al., 2013; Schröter et al., 2015; Wigger et al., 2017; Chazaud et al., 2006; Yamanaka et al., 2010; Wicklow et al., 2014), Lif/Stat (Morgani and Brickman, 2015; Onishi and Zandstra, 2015), Nodal/Smad2/3 (Mesnard et al., 2006; Papanayotou and Collignon, 2014), Bmp4/Smad4 (Graham et al., 2014; Wang et al., 2004) and Wnt/β-catenin (Corujo-Simon et al., 2017; ten Berge et al., 2011) pathways]. The initial PrE cell specification is reinforced by Epi inductions made through FGF4 signaling (Mulvey et al., 2015; De Caluwé et al., 2019; Molotkov and Soriano, 2018; Artus et al., 2013; Frum and Ralston, 2015; Houston, 2017) to progressively lock cell fates, to promote their physical segregation, and to promote the epithelialization and lining of the PrE along the blastocoel cavity (Meilhac et al., 2009; Burtscher and Lickert, 2009; Saiz et al., 2013; Brimson, 2016). This process is regulative as it senses and adjusts the mutually allocated cell numbers (Plusa and Hadjantonakis, 2018; Grabarek et al., 2012; Mathew et al., 2019; Yamanaka et al., 2010). The use of microsystems to control cell numbers (Vrij et al., 2016a) and of chemically defined medium (Kubaczka et al., 2014) opens possibilities to increase the control, throughput and screening capacities of embryo models (Vrij et al., 2016a; Rivron et al., 2018a).

By running combinatorial screens of proteins, GPCR ligands and small molecules in a microwell array platform and in chemically defined conditions ESCs can be directed to rapidly and efficiently co-form blastocyst-stage PrE-and Epi-like cells. These cells then develop synergistically in minimal medium to form a structure resembling the post-implantation Epi and extra-embryonic endoderm tissues (XEn), referred to as Epi/XEn. Mutual inductions between the Epi and PrE support the potential for growth, viability, specification and morphogenesis that underlie aspects of post-implantation development. The structure, so-called Rosette, contains a polarized epiblast-like (Epi) structure with a pro-amniotic cavity in the centre. The epiblast-like structure is enveloped by a polarized extraembryonic endoderm-like (ExEn) layer.

A particular aim of the invention is the development of a multicellular model that mimics key aspects of early embryonic development by co-induction of epiblast and extraembryonic endoderm to provide 3D cell structures having endoderm cells, mesoderm cells and ectoderm cells that can be maintained for a prolonged period and recapitulates aspects of organogenesis.

In a first aspect herein there is provided an in vitro method for producing a 3D structure of endoderm cells, mesoderm cells and ectoderm cells and capable of further organogenesis to form an organ or organ-like cell structure, the method comprising seeding one or more pluripotent stem cells in a culture chamber containing culture medium, culturing the seeded cell or cells in culture medium comprising an amount of chemical inducers for forming a plurality of pluripotent stem cells and inducing part of the formed pluripotent stem cells to form extraembryonic stem cells to obtain a primitive endoderm/epiblast aggregate, culturing the primitive endoderm/epiblast aggregate in fresh culture medium to obtain an extraembryonic endoderm/epiblast rosette structure, flushing the culture medium from the extraembryonic endoderm/epiblast rosette structure and culturing the extraembryonic endoderm/epiblast rosette structure in fresh serum free culture medium comprising a reducing agent to obtain the 3D structure of cells.

The starting cell population has a number of cells per culture chamber of between 1-500 cells, preferably between 5-100 and most preferably between 10-35. This starting cell population is first expanded in a medium that promotes a naïve pluripotency cell identity. Preferably, a medium based on the “2i/lif” conditions as described by Ying et al., 2008. Cells are seeded in culture chambers, preferably in microwells with a diameter of 50-25.000 μm (preferably 200-500 μm). The microwells are preferably conical or cylindrical shaped microwells.

Once the primitive endoderm/epiblast aggregate is established in the culture chamber, the structure may be progressed, in fresh basic culture medium without addition of exogenous signaling pathway modulators, to an embryo-like structure resembling the post-implantation extraembryonic endoderm/epiblast rosette structure. This rosette structure differs from the natural embryo in that it comprises a spheroidal radial symmetric structure of a pro-amniotic cavity surrounded by a polarized Epi covered with extraembryonic endoderm-like cells, instead of a half-spheroidal cup-shaped structure in natural embryos. Usually, in natural embryos, the other adjacent half-spheroidal cup-shaped structure consist of cells originating from trophoblast tissue, which also are believed to be critical for patterning further development of the embryo.

Surprisingly, a 3D structure of cells having endoderm cells, mesoderm cells and ectoderm cells can be obtained by flushing the culture medium from the extraembryonic endoderm/epiblast rosette structure and culturing the extraembryonic endoderm/epiblast rosette structure in fresh serum free culture medium comprising a reducing agent.

The fresh serum free medium supplemented with an oxygen scavenger not only permits the rosette structure to be maintained for prolonged periods of time, but to actually further develop into structures having endoderm cells, mesoderm cells and ectoderm cells and capable of further organogenesis to form an organ or organ-like cell structure. Particularly, the epiblast compartment within the extraembryonic endoderm/epiblast rosette structure is capable of differentiating into cells of the three germlayers ectoderm, endoderm, and mesoderm, with continued culture leading to differentiation of cells from the three germ layers. Hypoxia signals may partly or wholly contribute to induction of the germ layer mesoderm, ectoderm and endoderm differentiation within the structure. For example, using the method of the first aspect 3D structures of endoderm cells, mesoderm cells and ectoderm cells are obtained that can be further developed into cell tissues having a beating heart region.

There are not many complex 3D in vitro models that allow to mimic organogenesis in a dish. The multicellular model obtained by the method of the first aspect demonstrates inter-organ interaction during growth as several different embryonic tissues and organs develop simultaneously. The presence of multiple developing tissues and organs in the model enables a more systemic testing of compounds, e.g., perturbing the self-organization of the system, similar to a complex living embryo. The presence of extraembryonic endoderm tissue in the model helps patterning primordial tissues leading to organogenesis, which is an additional layer of complexity compared to e.g. known gastruloid models. Since no trophoblast tissue is present in this model, there are no or less ethical constraints in using the 3D structures.

The fresh serum free culture medium used in the method is a chemically-defined and xeno/serum-free cell culture medium, with a composition comprising a basal culture medium with B27 and N2 supplements. The basal medium can be a mixture of Dulbecco's Modified Eagle Medium/Nutrient Mixture F-12 (DMEM/F12) or Advanced DMEM/F12 or DMEM alone or CMRL-1066 (Invitrogen or Sigma), together with Neurobasal® medium. Preferably a mixture of advanced DMEM/F12 with Neurobasal in volume ratio 1:1. The B27 and N2 supplements in the medium are preferably in an amount between 0.25 and 1% of N2 and 0.5-2% B27. The B27 and N2 supplements may optionally be replaced with SOS® (Cat No M09-50, Cell Guidance Systems). Other neuronal supplements are also available and would be expected to be effective, as would be understood by a person skilled in the art. The culture medium may further comprise one or more of transferrin, selenium (for example sodium selenite), and/or ethanolamine, and/or an analogue thereof. Preferably, the culture medium comprises transferrin, selenium (for example sodium selenite) and ethanolamine. For example, the culture medium may comprise ITS-X (Invitrogen, 51500-056). The concentration of transferrin, or an analogue thereof, is preferably about 5.5 mg/L. The concentration of selenium (for example sodium selenite), or an analogue thereof, in the culture medium may preferably be about 0.0067 mg/L. The concentration of ethanolamine, or an analogue thereof, in the culture medium may be preferably about 2 mg/L. The culture medium may comprise L-glutamine or Glutamax at a concentration preferably about 2 mM. The culture medium may comprise sodium pyruvate at a concentration preferably of about 1 mM. The culture medium may comprise one, more than one or all components selected from the group consisting of L-glycine, L-alanine, L-asparagine, L-aspartic acid, L-glutamic acid, L-proline and L-serine. Preferably, the culture medium may comprise L-glycine at a concentration of about 7.5 mg/L, L-alanine at a concentration of about 9 mg/L, L-asparagine at a concentration of about 13 mg/L, L-aspartic acid at a concentration of about 13 mg/L, L-glutamic acid at a concentration of about 14.5 mg/L, L-proline at a concentration of about 11.5 mg/L and L-serine at a concentration of about 10.5 mg/L. The culture medium may contain glucose, for example added D-(+)-glucose, of preferably between 1 and 8 g/L. Optionally, oestrogen, an analogue thereof or an oestrogen receptor agonist, and/or progesterone, an analogue thereof or a progesterone receptor agonist can be added to the medium. The culture medium may further comprise non-essential amino acids (e.g. comprising glycine (e.g. about 5 mg/L to about 10 mg/L), L-alanine (e.g. about 5 mg/L to about 10 mg/L), L-asparagine (e.g. about 10 mg/L to about 15 mg/L), L-aspartic acid (e.g. about 10 mg/L to about 15 mg/L1), L-glutamic acid (e.g. about 10 mg/L to about 20 mg/L), L-proline (e.g. about 10 mg/L to about 15 mg/L) and/or L-serine (e.g. about 10 mg/L to about 15 mg/L).

In a particular embodiment of the method the culture medium comprises an amount of chemical inducers for forming a plurality of pluripotent stem cells from the one or more pluripotent stem cells and inducing part of the formed pluripotent stem cells to form extraembryonic stem cells, the one or more chemical inducers being selected from:
i) a retinoic acid receptor agonist and/or retinoid X receptor agonist and/or precursors thereof; ii) a cAMP analogue; iii) a FGF receptor agonist; and iv) a GSK3 inhibitor.

Optionally, the culture medium comprises an amount of a retinoic acid receptor agonist and/or a retinoid X receptor agonist and/or precursors thereof, wherein the agonist and/or precursor is one or more of retinoic acid, fenretinide, AR7, Adapalene, Acitretin, Tazarotene, Bexarotene, AM580, AR7, SR11237, TTNPB, Etretinate, All-trans-retinal, retinol, tretinoin, vitamin A, and preferably all-trans retinoic acid.

Optionally, the culture medium comprises an amount of a cAMP analogue, wherein the analogue is 8Br-cAMP.

Optionally, the culture medium comprises an amount of a FGF receptor agonist, wherein the agonist is selected from one or more of FGF2 and FGF4.

Optionally, the culture medium comprises an amount of a GSK3 inhibitor, wherein the inhibitor is selected from one or more of CHIR99021, BIO, BIO-acetoxime, and TWS119.

The reducing agent in the medium is preferably one or more selected from 2-mercaptoethanol (2-ME), (ß-mercaptoethanol), N-acetyl-L-cysteine, glutathione and dithiothreitol or any other suitable reducing agent. The concentration of the reducing agent in the culture medium is preferably about 100 μM.

In an embodiment of the method the culture medium comprising an amount of chemical inducers for forming a plurality of pluripotent stem cells and inducing part of the formed pluripotent stem cells to form extraembryonic stem cells is added to the one or more pluripotent stem cells between 0-72 hours after seeding the one or more pluripotent stem cells.

Optionally, the extraembryonic endoderm/epiblast rosette structure is cultured for a time period between 48-600 hours to obtain the 3D structure of cells.

In an embodiment of the method the culture chamber is formed by a microwell having a predefined shape configured to form the 3D structure of cells with desired shape.

Preferably, in the method the culture medium at least during the culturing of the extraembryonic endoderm/epiblast rosette structure is provided to the cells with convection, preferably by microfluidic flow of the culture medium and/or active agitation of the culture chamber.

In a particular embodiment of the method the serum free culture medium comprises an amount of one or more of:
i) an agonist, and/or a co-activator and/or an antagonist of the Activin type 1 receptor or Activin type 2 receptor;
ii) an agonist of the TGFbeta receptor;
iii) a modulator of the BMP receptor family and/or a BMP pathway antagonist;
iv) a signaling co-factor for the TGFbeta superfamily;
v) a Wnt signaling agonist;
vi) a retinoic acid pathway agonist;
vii) insulin, and/or an insulin/PI3k pathway activator, and/or an insulin receptor agonist, and/or an insulin analogue;
viii) a LIF/stat3 signaling activator;
ix) a FGF receptor agonist;
x) an EGF receptor agonist; and
xi) a molecule that increases intracellular cAMP levels and downstream signaling activation.

Preferably, the serum free culture medium comprises an amount of an agonist of the Activin type 1 or 2 receptor, wherein the agonist is Nodal or Activin A. In particular, Nodal may influence the extraembryonic specification ((Niakan et al., 2013; Mesnard et al., 2006) and visceral endoderm specification during the peri-implantation stage of development (Mesnard et al., 2006; Edgar et al., 2013; Pfister et al., 2007). Preferably the agonist is added to the medium in a concentration between 10 and 100 ng/ml.

Optionally, the serum free culture medium comprises an amount of a co-activator of the Activin type 1 or 2 receptor, wherein the co-activator is GDF3.

Optionally, the serum free culture medium comprises an amount of antagonist of the Activin type 1 or 2 receptor, wherein the antagonist is Cerberus, Lefty1, Cripto, or Follistatin.

Preferably, the serum free culture medium comprises an amount of an agonist of the TGFbeta receptor, wherein the agonist is TGFbeta1,2 or 3. Signals from the TGFß superfamily might induce the production of the basal lamina that serves as a base for epithelialization of the epiblast or extraembryonic endoderm (Moore et al., 2014).

Optionally, the serum free culture medium comprises an amount of a modulator of the BMP receptor family, wherein the modulator is BMP2, BMP4, BMP5, BMP7, or BMP8. Together with Wnt signals, BMP signals coming from the extraembryonic ectoderm, which is of trophoblast origin, likely trigger Wnt signals that in turn trigger gastrulation in the epiblast (anterior/posterior patterning).

Optionally, the serum free culture medium comprises an amount of a BMP pathway antagonist, wherein the antagonist is Noggin, Gremlin1, or Cerberus.

In an embodiment of the method the serum free culture medium comprises an amount of a signaling co-factor for the TGFbeta superfamily, wherein the co-factor is PCSK6, or Furin.

Optionally, the serum free culture medium comprises an amount of a Wnt signaling agonist, wherein the agonist is Wnt3a or a soluble Wnt analogue.

Optionally, the serum free culture medium comprises an amount of a GSK 3 inhibitor, wherein the inhibitor is Chir 99021, 6-bromoinsilvin-3′-oxime, or Kenpaullone.

Optionally, the serum free culture medium comprises an amount of a Wnt co-activator, wherein the co-activator is Hhex, or Sfrp5.

Optionally, the serum free culture medium comprises an amount of a Wnt antagonists, wherein the antagonist is Dikkopf-related protein 1, or Sfrp 1/5.

In an embodiment of the method the serum free culture medium comprises an amount of a retinoic acid pathway agonist, wherein the agonist is trans-retinoic acid, retinol, retinol-acetate, or all trans-retinol. Anterior-posterior specification of the Hox-gene pattern is influenced by retinoic acid signalling, which is expressed during gastrulation around the primitive streak (Corral en Storey 2004). Retinoic acid signaling is also involved in limb positioning and anterior/posterior patterning of the endoderm.

Optionally, the serum free culture medium comprises an amount of an insulin receptor agonist, wherein the agonist is IGF1, IGF2. The insulin receptor agonist and insulin/PI3k pathway activator are involved in embryo growth. Preferably, the agonist is added to the culture medium in a concentration of between 0.05 ng/ml to about 300 ng/ml, preferably between about 25 ng/ml to about 75 ng/ml.

Optionally, the serum free culture medium comprises an amount of a LIF/stat3 signaling activator, wherein the activator is one or more of leukemia inhibitory factor (LIF), IL6, IL11, IL-27, IL-31, oncostatin M, cardiotrophin-1, neuropoietin, or cardiotrophin-like cytokine 1. Lif/Stat3 signalling is essential for early post-implantation development of the embryo; stat3 negative embryos stop developing after gastrulation. Trophoblast cells are known to release Lif that activates Stat3.

Optionally, the serum free culture medium comprises an amount of a FGF receptor agonist, wherein the agonist is bFGF/FGF2, FGF4, FGF5, or FGF8. FGFs are factors released by the extraembryonic tissues that help regulate patterning of the embryo.

Optionally, the serum free culture medium comprises an amount of a FGF receptor co-factor, wherein the co-factor is FRS2.

Optionally, the serum free culture medium comprises an amount of a EGF receptor agonist, wherein the agonist is hb-EGF. EGF agonist can activate Stat3.

Optionally, the serum free culture medium comprises an amount of an Adenylyl cyclase modulator, wherein the modulator is Forskolin or an analogue of cAMP such as 8br-cAMP. cAMP/PKA signaling is critical for heart tube formation.

In a particular embodiment of the method the extraembryonic endoderm/epiblast rosette structure is cultured in fresh serum free culture medium comprising a reducing agent until a 3D structure of endoderm cells, mesoderm cells and ectoderm cells with spontaneously contracting region resembling a developing embryonic heart is obtained.

In a second aspect herein a culture medium comprising an amount of chemical inducers for forming a plurality of pluripotent stem cells from the one or more pluripotent stem cells and inducing part of the formed pluripotent stem cells to form extraembryonic stem cells as described herein for use in the method of the first aspect is provided.

In a third aspect herein a serum free culture medium as described herein for use in the method of the first aspect is provided.

In order to enable a continued and extended growth and development of the 3D multicellular construct capable of organogenesis, a transfer of the construct from a static to a dynamic cell culture system is favorable. This transfer can be carried out on any day starting from the seeding on day 0 and should preferably be done before day 6. The dynamic cell culture system for the biological constructs is a miniaturized and microfluidic cell culture chamber, here referred to as microbioreactor, in which various cell culture parameters can be measured and precisely controlled. Because of the miniaturization, the microbioreactor can easily be scaled up in a parallel manner in order to increase throughput.

The design of the microbioreactor can be based on irreversible and reversible sealing concepts. The materials used to fabricate the bioreactor will be either polymers (thermoplastics, thermoplastic elastomers or elastomers), metals, glass or ceramics.

The active flow in the microbioreactor can be either a continuous or a discontinuous flow. This includes also concepts of multiphase microfluidics, e.g., droplet microfluidics, where the biological construct is embedded in a smaller compartment harboring a biocompatible phase and which is separated from other compartments, harboring also biological constructs, by a second liquid phase (e.g. hydrophilic and hydrophobic phase, such as oil-water-based systems and systems based on differences in viscosity).

A central task of the microbioreactor is to provide a defined dynamic environment where the supply of nutrients and gases (partial pressure of O₂, N₂, CO₂ and other gases) and the mechanical forces acting on the biological construct can be dynamically controlled. In a favorable design, the microbioreactor allows a complete decoupling of these parameters, e.g., by shielding the biological construct from stress by culturing them in confinements, such as microwells. The concentration/partial pressures of the gases and the soluble factors in the medium will be defined in the medium reservoir, which is fluidically connected to the microbioreactor. The pumps will actively displace the medium from the reservoir and pushes or pulls it through the microbioreactor back into the medium reservoir (closed loop). In an alternative scenario, the medium coming from the microbioreactor is collected in a waste compartment, from which, e.g., samples of the medium can be taken (continuously/discontinuously).

The pumps for displacing the medium in the cell culture system can be either pressure- (e.g., driven by gases, electrical or magnetic fields, gravitation) or flow-controlled (e.g., syringe pumps, peristaltic/roller pumps) systems. Another additional feature of the microbioreactor is the possibility to switch between different flow modes, i.e. real and forced perfusion of medium through the biological construct (perfusion) or flow around the whole or parts of the surface area of the construct (superfusion). The flow direction in the microbioreactor can be uni-, bi- or multidirectional, which also allows exposing some parts of the biological construct to higher or lower/no shear flow than other parts. The directions of flow can dynamically be changed by integrated valves in the fluidic periphery of the microbioreactor (e.g., in the tubing system). Depending on the connected type of microfluidic pump, e.g., syringe or peristaltic/roller pump, the flow is smooth without any pressure changes over time or, to mimic blood flow at later stages of organogenesis, pulsating. In the latter case, computer-controlled pumps can fully control amplitude, frequency and damping of the flow pulses in the microbiorector.

In order to observe the biological construct during the culture, parts of the microbioreactor (housing) are transparent. This allows a continuous monitoring and imaging of the biological specimen. To further control the culture of the biological construct, the construct is, at least temporarily, fixed and/or controlled in its position/rotation by embedding it in a hydrogel, embedding it in a microwell/milliwell or tray (with a defined geometry and shape), embedding it in a porous microwell/milliwell or tray (with a defined geometry and shape), or entrapping it in microfluidic compartments or chambers with a defined shape and size (this also includes droplet-based compartments with higher viscosity or different phases).

In a most favorable design, the microbioreactor allows the integration of at least one sensor or sensor combination to monitor cell culture relevant physical and biochemical parameters in real-time. This includes for example O₂- and CO₂-concentrations, temperature, pH, glucose concentration, concentrations of medium components or molecules secreted from the biological construct, such as growth factors. Further analytical techniques, such as HPLC or mass spectrometry, can be integrated in-line in a closed- or open-loop configuration.

It will be clear to the skilled person that the invention is not limited to any embodiment herein described and that modifications are possible which should be considered within the scope of the appended claims. Also kinematic inversions are considered inherently disclosed and to be within the scope of the invention. In the claims, any reference signs shall not be construed as limiting the claim. The term ‘comprising’ and ‘including’ when used in this description or the appended claims should not be construed in an exclusive or exhaustive sense but rather in an inclusive sense. Thus the expression ‘comprising’ as used herein does not exclude the presence of other elements or steps in addition to those listed in any claim. Furthermore, the words ‘a’ and ‘an’ shall not be construed as limited to ‘only one’, but instead are used to mean ‘at least one’, and do not exclude a plurality. Features that are not specifically or explicitly described or claimed may be additionally included in the structure of the invention within its scope. Expressions such as: “means for . . . ” should be read as: “component configured for . . . ” or “member constructed to . . . ” and should be construed to include equivalents for the structures disclosed. The use of expressions like: “critical”, “preferred”, “especially preferred” etc. is not intended to limit the invention. Additions, deletions, and modifications within the purview of the skilled person may generally be made without departing from the spirit and scope of the invention, as is determined by the claims.

1. An in vitro method for producing a 3D structure of endoderm cells, mesoderm cells and ectoderm cells and capable of further organogenesis to form an organ or organ-like cell structure, the method comprising seeding one or more pluripotent stem cells in a culture chamber containing culture medium, culturing the seeded cell or cells in culture medium comprising an amount of chemical inducers for forming a plurality of pluripotent stem cells and inducing part of the formed pluripotent stem cells to form extraembryonic stem cells to obtain a primitive endoderm/epiblast aggregate, culturing the primitive endoderm/epiblast aggregate in fresh culture medium to obtain an extraembryonic endoderm/epiblast rosette structure, flushing the culture medium from the extraembryonic endoderm/epiblast rosette structure and culturing the extraembryonic endoderm/epiblast rosette structure in fresh serum free culture medium comprising a reducing agent to obtain the 3D structure of cells.

2. The method according to claim 1, wherein the culture medium comprising an amount of chemical inducers for forming a plurality of pluripotent stem cells and inducing part of the formed pluripotent stem cells to form extraembryonic stem cells comprises one or more chemical inducers selected from:
i) a retinoic acid receptor agonist and/or retinoid X receptor agonist and/or precursors thereof;
ii) a cAMP analogue;
iii) a FGF receptor agonist; and
iv) a GSK3 inhibitor.

3. The method according to claim 2, wherein:
A. the culture medium comprising an amount of chemical inducers for forming a plurality of pluripotent stem cells and inducing part of the formed pluripotent stem cells to form extraembryonic stem cells comprises an amount of a retinoic acid receptor agonist and/or a retinoid X receptor agonist and/or precursors thereof, wherein the agonist and/or precursor is one or more of retinoic acid, fenretinide, AR7, Adapalene, Acitretin, Tazarotene, Bexarotene, AM580, AR7, SR11237, TTNPB, Etretinate, All-trans-retinal, retinol, tretinoin, and vitamin A; and/or
B. wherein the culture medium comprising an amount of chemical inducers for forming a plurality of pluripotent stem cells and inducing part of the formed pluripotent stem cells to form extraembryonic stem cells comprises an amount of a cAMP analogue, wherein the analogue is 8Br-cAMP; and/or
C. wherein the culture medium comprising an amount of chemical inducers for forming a plurality of pluripotent stem cells and inducing part of the formed pluripotent stem cells to form extraembryonic stem cells comprises an amount of a FGF receptor agonist, wherein the agonist is selected from one or more of FGF2 and FGF4; and/or
D. wherein the culture medium comprising an amount of chemical inducers for forming a plurality of pluripotent stem cells and inducing part of the formed pluripotent stem cells to form extraembryonic stem cells comprises an amount of a GSK3 inhibitor, wherein the inhibitor is selected from one or more of CHIR99021, BIO, BIO-acetoxime, and TWS119.

4-6. (canceled)

7. The method according to claim 1, wherein the reducing agent is one or more selected from 2-mercaptoethanol (2-ME), ß-mercaptoethanol, N-acetyl-L-cysteine, glutathione and dithiothreitol.

8. The method according to claim 1, wherein the culture medium comprising an amount of chemical inducers for forming a plurality of pluripotent stem cells and inducing part of the formed pluripotent stem cells to form extraembryonic stem cells is added to the one or more pluripotent stem cells between 0-72 hours after seeding the one or more pluripotent stem cells.

9. The method according to claim 1, wherein the extraembryonic endoderm/epiblast rosette structure is cultured for a time period between 48-600 hours to obtain the 3D structure of cells.

10. The method according to claim 1, wherein the culture chamber is formed by a microwell having a predefined shape configured to form the 3D structure of cells with desired shape.

11. The method according to claim 1, wherein the culture medium at least during the culturing of the extraembryonic endoderm/epiblast rosette structure is provided to the cells with convection.

12. The method according to claim 1, wherein the serum free culture medium comprises an amount of one or more of:
i) an agonist, and/or a co-activator and/or an antagonist of the Activin type 1 receptor or Activin type 2 receptor;
ii) an agonist of the TGFbeta receptor;
iii) a modulator of the BMP receptor family and/or a BMP pathway antagonist;
iv) a signaling co-factor for the TGFbeta superfamily;
v) a Wnt signaling agonist;
vi) a retinoic acid pathway agonist;
vii) insulin, and/or an insulin/PI3k pathway activator, and/or an insulin receptor agonist, and/or an insulin analogue;
viii) a LIF/stat3 signaling activator;
ix) a FGF receptor agonist;
x) an EGF receptor agonist; and
xi) a molecule that increases intracellular cAMP levels and downstream signaling activation.

13. The method according to claim 12, wherein:
A. the serum free culture medium comprises an amount of an agonist of the Activin type 1 or 2 receptor, wherein the agonist is Nodal or Activin A; and/or
B. the serum free culture medium comprises an amount of a co-activator of the Activin type 1 or 2 receptor, wherein the co-activator is GDF3; and/or
C. the serum free culture medium comprises an amount of antagonist of the Activin type 1 or 2 receptor, wherein the antagonist is Cerberus, Lefty1, Cripto, or Follistatin; and/or
D. the serum free culture medium comprises an amount of an agonist of the TGFbeta receptor, wherein the agonist is TGFbeta1,2 or 3; and/or
E. the serum free culture medium comprises an amount of a modulator of the BMP receptor family, wherein the modulator is BMP2, BMP4, BMP5, BMP7, or BMP8; and/or
F. the serum free culture medium comprises an amount of a BMP pathway antagonist, wherein the antagonist is Noggin, Gremlin1, or Cerberus.

14-18. (canceled)

19. The method according to claim 12, wherein:
A. the serum free culture medium comprises an amount of a signaling co-factor for the TGFbeta superfamily, wherein the co-factor is PCSK6, or Furin; and/or
B. the serum free culture medium comprises an amount of a Wnt signaling agonist, wherein the agonist is Wnt3a or a soluble Wnt analogue; and/or
C. the serum free culture medium comprises an amount of a GSK3 inhibitor, wherein the inhibitor is Chir 99021, 6-bromoinsilvin-3′-oxime, or Kenpaullone; and/or
D. the serum free culture medium comprises an amount of a Wnt co-activator, wherein the co-activator is Hhex, or Sfrp5; and/or
E. the serum free culture medium comprises an amount of a Wnt antagonists, wherein the antagonist is Dickkopf-related protein 1, or Sfrp1/5; and/or
F. the serum free culture medium comprises an amount of a retinoic acid pathway agonist, wherein the agonist is trans-retinoic acid, retinol, retinol-acetate, or all trans-retinol.

20-24. (canceled)

25. The method according to claim 12, wherein the serum free culture medium comprises an amount of an insulin receptor agonist, wherein the agonist is IGF1, IGF2.

26. The method according to claim 12, wherein the serum free culture medium comprises an amount of a LIF/stat3 signaling activator, wherein the activator is one or more of leukemia inhibitory factor (LIF), IL6, IL11, IL-27, IL-31, oncostatin M, cardiotrophin-1, neuropoietin, or cardiotrophin-like cytokine 1.

27. The method according to claim 12, wherein the serum free culture medium comprises an amount of a FGF receptor agonist, wherein the agonist is bFGF/FGF2, FGF4, FGF5, or FGF8.

28. The method according to claim 12, wherein the serum free culture medium comprises an amount of a FGF receptor co-factor, wherein the co-factor is FRS2.

29. The method according to claim 12, wherein the serum free culture medium comprises an amount of a EGF receptor agonist, wherein the agonist is hb-EGF.

30. The method according to claim 12, wherein the serum free culture medium comprises an amount of an Adenylyl cyclase modulator, wherein the modulator is Forskolin or an analogue of cAMP.

31. The method according to claim 1, wherein the extraembryonic endoderm/epiblast rosette structure is cultured in fresh serum free culture medium comprising a reducing agent to obtain the 3D structure of endoderm cells, mesoderm cells and ectoderm cells with spontaneously contracting region resembling a developing embryonic heart.

32. A culture medium comprising an amount of chemical inducers for forming a plurality of pluripotent stem cells and inducing part of the formed pluripotent stem cells to form extraembryonic stem cells, wherein said one or more chemical inducers are selected from:
i) a retinoic acid receptor agonist and/or retinoid X receptor agonist and/or precursors thereof;
ii) a cAMP analogue;
iii) a FGF receptor agonist; and
iv) a GSK3 inhibitor.
